(19) United States
(12) Patent Application Publication
(10) Pub. No.: US 2025/0311921 A1
(43) Pub. Date: Oct. 9, 2025

(54) SYSTEMS AND DEVICES FOR DRY EYE DETECTION, PREVENTION, AND DIAGNOSIS

(71) Applicant: ALCON INC., Fribourg (CH)

(72) Inventor: Armin Parsa, Newport Coast, CA (US)

(21) Appl. No.: 19/085,411

(22) Filed: Mar. 20, 2025

Related U.S. Application Data
(60) Provisional application No. 63/574,586, filed on Apr. 4, 2024.

(51) Int. Cl. A61B 3/10

(52) U.S. Cl. CPC A61B 3/101

(57) ABSTRACT

Devices, systems, and methods for monitoring tear film thickness are disclosed. In certain embodiments, a device includes a lens and one or more electronic components coupled to the lens. The one or more electronic components are operable to receive a first signal and transmit a second, return signal associated with an electrical characteristic of the one or more components.

### INTRODUCTION

This section provides information to facilitate a better understanding of the various aspects of the disclosure. It should be understood that the statements in this section of this document are to be read in this light, and not as admissions of prior art.

Dry eyes may result from a variety of factors, such as, age, illness, poor blinking habits, dry environments, medications, the use of contact lenses, prolonged computer, tablet and/or phone use, combinations of the same, and the like. Dry eye may be associated with the volume and/or thickness of the tear film, or specific layers of the tear film. The tear film is a protective layer covering the ocular surface of an eye and provides lubrication to the eye. The tear film has three layers that include the outer lipid layer, the middle aqueous layer, and the inner mucin layer. The inner layer provides stability and allows for tears to attach to the cornea. The middle aqueous layer contains electrolytes, proteins, and antibodies to protect the eye from infections and debris. The outer lipid (or oil) layer slows down the rate of evaporation of the tear film.

However, current methods of measuring the tear film generally require visiting an optometrist which can be time consuming and/or expensive. Additionally, current methods only provide data relating to the tear film thickness at a single point in time.

### SUMMARY

This summary is provided to introduce a selection of concepts that are further described below in the Detailed Description. This summary is not intended to identify key or essential features of the claimed subject matter, nor is it to be used as an aid in limiting the scope of the claimed subject matter.

In one or more embodiments, a device for facilitating monitoring of tear film thickness is disclosed. The device includes a lens and one or more electronic components coupled to the lens. The one or more electronic components are operable to receive a first wireless signal from an external device and, responsive to the first wireless signal, transmit a second wireless signal associated with an electrical characteristic of the one or more electrical components. In some embodiments, the external device is operable to receive the second wireless signal and determine the tear film thickness based on the second signal.

In one or more embodiments, a system for monitoring tear film thickness is disclosed. The system includes an external device operable to transmit a first wireless signal, a lens, and one or more electronic components coupled to the lens. The one or more electronic components are operable to receive the first wireless signal from the external device and, responsive to the first wireless signal, transmit a second wireless signal associated with an electrical characteristic of the one or more electrical components. In some embodiments, the external device is operable to receive the second wireless signal and determine the tear film thickness based on the signal.

In one or more embodiments, a method of monitoring tear film thickness is disclosed, the method includes sending a first wireless signal to a device operable to facilitate monitoring the tear film thickness, the device comprising a lens and one or more electrical components coupled to the lens. The method further includes responsive to the first wireless signal, receiving a second wireless signal transmitted by the device, wherein the second wireless signal is associated with an electrical characteristic associated with the one or more electrical components. The method further includes determining the tear film thickness of the eye based on the second signal.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates an example device for monitoring tear film thickness, according to certain embodiments.

FIG. 2 illustrates a second example device for monitoring tear film thickness, according to certain embodiments.

FIG. 3 illustrates a third example device for monitoring tear film thickness, according to certain embodiments

FIG. 4A illustrates an example system for monitoring tear film thickness, according to certain embodiments.

FIG. 4B illustrates an example external device, according to aspects of the disclosure.

FIG. 5 illustrates an example method for monitoring tear film thickness, according to certain embodiments.

FIG. 6 illustrates an example method of monitoring tear film thickness, according to certain embodiments.

FIG. 7A illustrates an example tear film thickness monitoring system, according to aspects of the disclosure.

FIG. 7B illustrates an infrared (IR) temperature sensor integrated into a phone, according to aspects of the disclosure.

FIG. 7C illustrates an augmented reality and/or virtual reality (AR/VR) headset having IR temperature sensors disposed on lenses, according to aspects of the disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Embodiments described herein provide systems and methods for monitoring tear film thickness on a more frequent (e.g., continuous) basis. For example, certain embodiments herein provide a device including a lens and an electronic component whose electrical characteristic is associated with, or indicative of, a tear film thickness of an eye. The electrical characteristic of the one or more electric components can be monitored over time to allow for monitoring the tear film thickness. For example, the device may transmit a signal reflective of the electrical characteristic to an external device, which can then map the signal to the tear film thickness of the eye. In various embodiments, the one or more electronic components can include one or more antennas, a capacitive element, a temperature sensor, or any combination thereof.

This continuous monitoring of the tear film thickness (e.g., in defined time increments such as milliseconds (e.g., 5, 10, 20, etc. milliseconds (ms)), seconds (e.g., 5, 10, 20, etc. seconds), minutes (e.g., 5, 10, 20, etc. minutes), etc.) enables provision of real time alerts to the user of the external device, for example, when the change in the tear film thickness is larger than a defined threshold or when the tear film thickness drops below a threshold thickness, thereby allowing the user to stop or prevent dry eye before irritation occurs. Further, monitoring the tear film thickness over time allows for identifying patterns that can be analyzed to determine, for example, a root cause associated with times during the day where dry eye develops, or potential times that would be optimal for the user to use topical treatments for the eye (e.g., eye drops). Furthermore, tear film data developed as a result of this continuous monitoring may be uploaded or transmitted to a doctor and/or clinician to assist in the diagnosis of dry eye.

While described as occurring at a regular interval (e.g., every 10 ms), it will be appreciated that the same principles are equally applicable to monitoring the tear film thickness using a triggering approach (e.g., when a user blinks as a triggering event) or a hybrid approach (e.g., measuring tear film thickness in a burst of readings every 300 ms for four measurements after a blink, and another burst of four readings every 5 seconds afterwards completion of the initial burst without another triggering event).

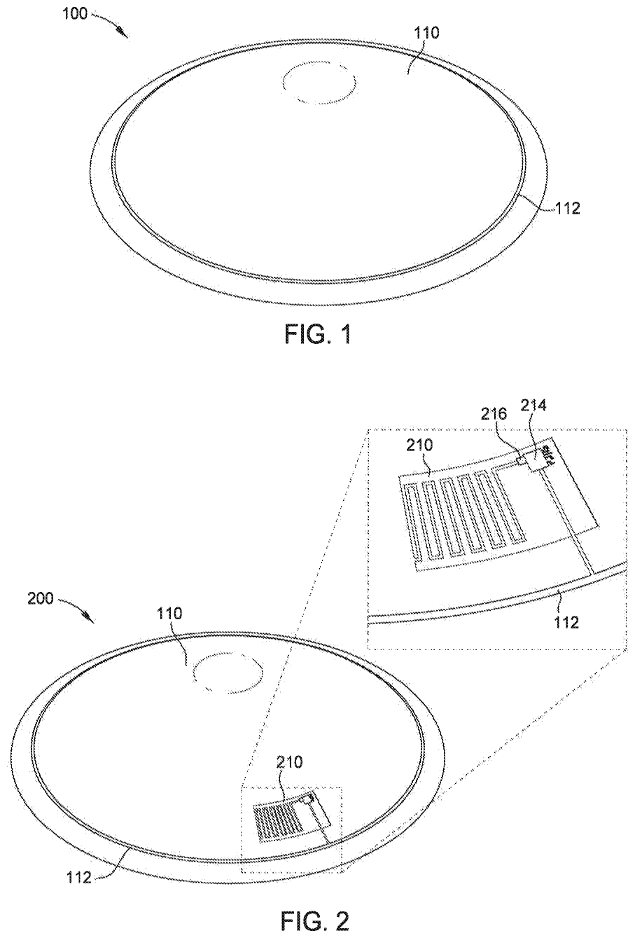

FIG. 1 illustrates an example device 100 for monitoring tear film thickness, according to certain embodiments. The device 100 includes a lens 110 coupled to one or more electronic components. In some embodiments, lens 110 may be a rigid gas permeable lens, a soft lens, a disposable lens, an extended wear lens, or any other type of contact and/or decorative lens shaped and/or configured to be placed on the eye of a user or patient to monitor the tear film thickness of the patient over a given period of time. The one or more electronic components may be coupled, for example, to an outside surface of the lens 110 (e.g., the surface opposite where the device 100 contacts the eye). In the example of FIG. 1, the one or more electronic components coupled to the lens 110 includes an antenna 112.

In certain embodiments, tear film thickness may be proportional to a resonance frequency of the antenna 112. In particular, a resonance frequency shift over time may occur in response to a change to the thickness of the tear film. Therefore, the tear film thickness can be monitored over time based on monitoring the resonance frequency of the antenna 112. For example, in the example device 100 of FIG. 1, the electrical characteristic used to monitor the tear film thickness may be the resonance frequency of the antenna 112. To allow for monitoring the corresponding tear film thickness, the antenna 112 can be configured to transmit a signal (e.g., an electromagnetic wave) including or indicative of the resonance frequency to an external device. For example, the antenna 112 may be operable to receive a first signal from the external device, for example a smart phone, and responsive to the first signal, the antenna 112 transmits a second signal (also referred to herein as a “return signal”) back to the external device. The return signal is indicative of a resonance frequency of the antenna 112, which the external device can map to a tear film thickness of the eye. As an example, if the tear film thickness falls outside a desired range, the external device can then notify the user.

In certain embodiments, to determine the resonance frequency of the antenna 112 at a given time, the external device may transmit a plurality of first signals at varying frequencies to the antenna 112. Responsive to each of the plurality of first signals, the antenna 112 may transmit a return signal back to the external device. The external device may then process these return signals to determine at which frequency the antenna 112 exhibits a maximum oscillatory response, which corresponds to the resonance frequency of the antenna 112. This determined frequency of the antenna 112 may then be mapped by the external device to a tear film thickness of the eye that is proportional to the resonance frequency.

In certain embodiments, the external device may calculate a change in the resonance frequency of the antenna 112 using two resonance frequencies at two different points in time (T0 and T1). The change or shift in the resonance frequency, as described above, corresponds to a change in the tear film thickness. As an example, if the change in the tear film thickness falls outside a desired range, the external device can then notify the user.

The antenna 112 may be located in a variety of locations in relation to the lens 110. For example, in some embodiments, the antenna 112 is disposed around a circumference of the lens 110. In another example, the antenna 112 is disposed around a partial circumference of the lens 110. In some embodiments, the antenna 112 may have a plurality of antenna arms. For example, each antenna arm may have a corresponding resonance frequency. The resonance frequencies of the antenna arms may then be used to provide an average resonance frequency associated with the tear film thickness. The average resonance frequency can then be mapped over time, which would reflect the thickness of the tear film over time. In some circumstances, the average resonance frequency obtained by averaging multiple resonance frequencies can provide a generally more accurate measurement of the tear film thickness.

In some embodiments, the antenna 112 is a passive antenna, such as a radio frequency identification (RFID) antenna, which only transmits a signal upon first receiving a signal from an external device. In some embodiments, the antenna 112 may include a surface acoustic wave (SAW) based antenna sensor. For example, a SAW-based sensor may use mechanical vibrations that propagate when surface acoustic waves are excited by an electrical signal at the resonance frequency of the antenna. The changes in the velocity of the SAW can then be correlated to changes in the tear film thickness. Additionally and/or alternatively, in certain embodiments, the electronic components may include one or more integrated circuit chips and/or one or more other sensors/sensor elements, as described below.

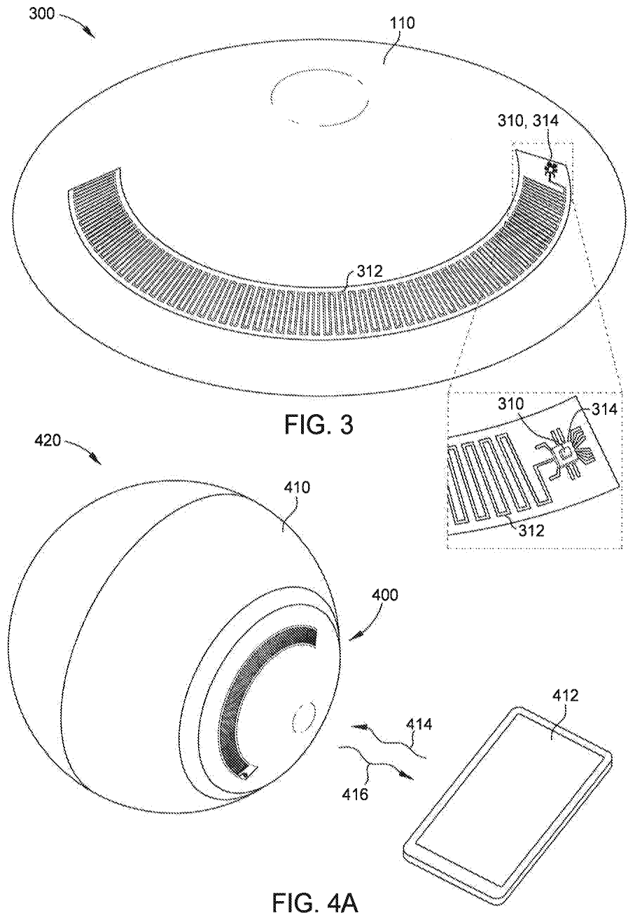

In some embodiments, the antenna 112 is further operably coupled to (e.g., electrically coupled to) a radio frequency integrated circuit chip (RFIC) also integrated into the lens 110 (e.g., see FIG. 2). In such embodiments, the RFIC includes a silicon semiconductor device chip and read-write memory. In some embodiments, the RFIC includes a passive, backscatter RFIC that harvests energy from the RF field of a nearby RFID reader via the antenna 112 and communicates by changing its impedance to modulate the amount of power transmitted back to the reader. In embodiments with an RFIC, the antenna 112 may be operable to receive a first signal from an external device and relay the first signal to the RFIC. Energy from the first signal can be harvested by the RFIC to process the relayed signal via signal matching at one or more frequencies, and then transmit a return signal to the antenna 112 for relay to the external device. Such return signal may be modulated by the RFIC to be indicative of the resonance frequency of the antenna 112, which corresponds to the tear film thickness of the eye. In some embodiments, the return signal includes data representative of the resonance frequency of the antenna 112, which can be encoded into the signal's modulation or digital payload for reading by the external device.

In certain embodiments, the antenna 112 may be, or may include, a passive three-port RFID sensing architecture-type antenna. For example, the passive three-port sensing architecture may include an RFIC and a sensing element (e.g., a sensor). The sensing element may convert detected inputs, which correspond to changes in the liquid level of the tear film, to corresponding fringe-capacitance variations, which alters the phase of an RFID backscattered signal (e.g., a return signal to an external device). The change is used as a sensing parameter by the RFID architecture for liquid level detection.

FIG. 2 illustrates an example device 200 for monitoring tear film thickness, according to certain embodiments. The device 200 includes the lens 110 described above relative to FIG. 1. In the example of FIG. 2, the one or more electronic components coupled to the lens 110 include the antenna 112 discussed above, as well as an RFIC 214 or other suitable type of chip, a capacitance sensor 216, and a capacitive element 210 (e.g., a capacitor). As shown, capacitive element 210 is electrically coupled to the capacitance sensor 216 and RFIC 214, which is electrically coupled to the antenna 112.

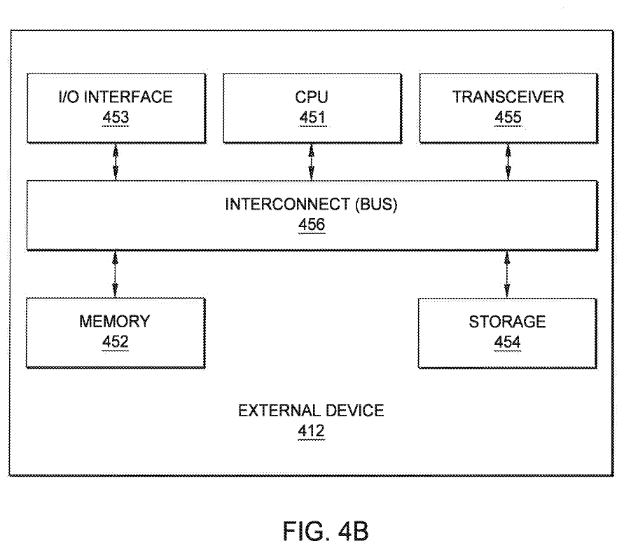

In the example of FIG. 2, the RFIC 214 includes one or more ports for external sensors, including the capacitance sensor 216. In some embodiments, however, a sensor can be integrated onto an RFIC itself (e.g., see FIG. 3). For example, the RFIC 214 itself may include the capacitance sensor 216.

In some embodiments, the capacitive element 210 may be disposed around a circumference of the lens 110. In certain embodiments, the capacitive element 210 may be disposed around a partial circumference of the lens 110. In some embodiments, the capacitive element 210 may match a similar geometry to the antenna 112. In some embodiments, the capacitive element 210 may include a plurality of capacitive elements 210. In some embodiments, the capacitive element 210 may include an interdigital capacitor. In certain embodiments, the capacitive element 210 may include a parallel plate capacitor, or two parallel plate capacitors plated or printed on different layers of the lens 110. In some embodiments, the capacitive element 210 may include hexagonal-shaped complementary split ring resonators (CSRRs), arranged in, for example, a honey-cell configuration.

In certain embodiments, tear film thickness may correspond to a capacitance of the capacitive element 210. In other words, a change in capacitance over time occurs in response to a change to the thickness of the tear film. Therefore, the tear film thickness can be monitored over time based on monitoring the capacitance of the capacitive element 210. And, as an example, if the tear film thickness falls outside a desired range, the external device can then notify the user. Thus, in certain embodiments, the electrical characteristic used to measure the tear film thickness may include a capacitance of the capacitive element 210, as detected by capacitance sensor 216.

In some embodiments, the antenna 112 may transmit a signal associated with, or indicative of, the capacitance of capacitive element 210 to an external device. For example, the antenna 112 can receive a first signal from the external device, for example a smart phone, and then energy from the first signal can be harvested by the RFIC 214 and/or capacitance sensor 216 to cause the capacitance sensor 216 to measure the capacitance of the capacitive element 210. The capacitance reading by the capacitance sensor 216 is then obtained by the RFIC 214, which then transmits a second, “return” signal to the antenna 112 to relay to the external device. This signal is modulated by the RFIC 214 to be indicative of the measured capacitance of the capacitive element 210, which corresponds to the tear film thickness of the eye. In some embodiments, the return signal includes data representative of the capacitance, which can be encoded into the signal's modulation or digital payload for reading by the external device.

In certain embodiments, the external device may calculate a change in the capacitance of the capacitive element 210 using two capacitance values at two different points in time (T0 and T1). The change in the capacitance, as described above, corresponds to a change in the tear film thickness. As an example, if the change in the tear film thickness falls outside a desired range, the external device can then notify the user.

In certain embodiments, a signal indicative of the resonance frequency of the antenna 112, as described in relation to FIG. 1, and a signal indicative the capacitance of the capacitive element 210, can both be transmitted to the external device, which may allow for a more accurate determination of the tear film thickness.

FIG. 3 illustrates an example device 300 for monitoring tear film thickness, according to certain embodiments. The device 300 includes the lens 110 described above relative to FIG. 1. In the example of FIG. 3, the one or more electronic components coupled to the lens 110 include a temperature sensor 310 operable to determine temperature at the surface of the eye. In some embodiments, the temperature sensor 310 may be disposed along a circumference of the lens 110. In some embodiments, the temperature sensor 310 may include a plurality of temperature sensors 310.

In some embodiments, the temperature sensor 310 may have, or be electrically coupled to, passive RFID components. For example, as illustrated in FIG. 3, the temperature sensor 310 may be integrated with an RFIC 314, which is coupled to an antenna component 312, such as the antenna 112. In such examples, the antenna component 312 may receive a signal from an external RFID device and transmit the signal to the RFIC 314 and sensor 310 to power up the RFIC 314 and sensor 310. In some examples, the temperature sensor 310 is separate from but electrically coupled to the RFIC 314 via, e.g., a port of the RFIC 314.

In certain embodiments, changes in the temperature of the eye, which can be measured by the temperature sensor 310, are reflective of the tear film thickness. In particular, evaporation of fluids in the eye may lead to temperature changes in the eye, and thus the magnitude of the temperature change after, for example a blink, could be indicative of the thickness of the tear film. As an example, a larger than average temperature change, could be indicative of a thinner than average tear film thickness. Accordingly, in certain embodiments, temperature at the surface of the eye, as sensed by the temperature sensor 310, may be used as a parameter for monitoring tear film thickness.

In some embodiments, the temperature sensor 310 may transmit a signal indicative of the temperature at the surface of the eye to an external device. For example, the antenna 312 can receive a first signal from the external device, for example a smart phone, and then the energy from the first signal can be harvested by the RFIC 314 and/or temperature sensor 310, causing the temperature sensor 310, in turn, to take a temperature reading. The temperature reading is then provided to the RFIC 314, and a modulated second, or return, signal from the RFIC 314 is transmitted to the antenna 312 to be relayed back to the external device. The modulated signal is indicative of the sensed temperature at the surface of the eye, which corresponds to the tear film thickness of the eye. In some embodiments, the return signal includes data representative of the sensed temperature, which can be encoded into the signal's modulation or digital payload for reading by the external device.

In certain embodiments, the external device may calculate a change in the temperature of the eye using two temperature values at two different points in time (T0 and T1). The change in the temperature, as described above, corresponds to the tear film thickness. As an example, if the tear film thickness falls outside a desired range, the external device can then notify the user.

In certain embodiments, a certain temperature value communicated by a signal transmitted by the temperature sensor 310 could be directly indicative of the tear film thickness of the eye. For example, the external device (e.g., RFID reader or mobile phone) may include a lookup table or other data structure that correlates temperatures values to tear film thicknesses. In such embodiments, the external device is configured to map the temperature received from the temperature sensor 310 to a certain tear film thickness value. In these and other embodiments, if the tear film thickness falls outside a desired range, the external device can then notify the user.

In some embodiments, in addition to the temperature sensor 310, the one or more electronic components of the lens 110 may further include, for example, the antenna 112 and/or the capacitive element 210. As described above, multiple electronic components may allow for a more accurate determination of the tear film thickness. For example, a change in temperature may be measured in addition to a change in capacitance and/or a change in resonance frequency. A mapping of the change in temperature, capacitance, and/or resonance frequency each provide a value associated with tear film thickness. In certain embodiments, the tear film thickness values derived from the multiple electrical components may then be used to provide an average tear film thickness.

While the example device 300 is described as including a temperature sensor 310 for sensing temperature at the surface of the eye, it is further contemplated that the example device 300 may alternatively or additionally include other types of sensors for sensing parameters of the eye other than temperature that may be indicative of tear film thickness.

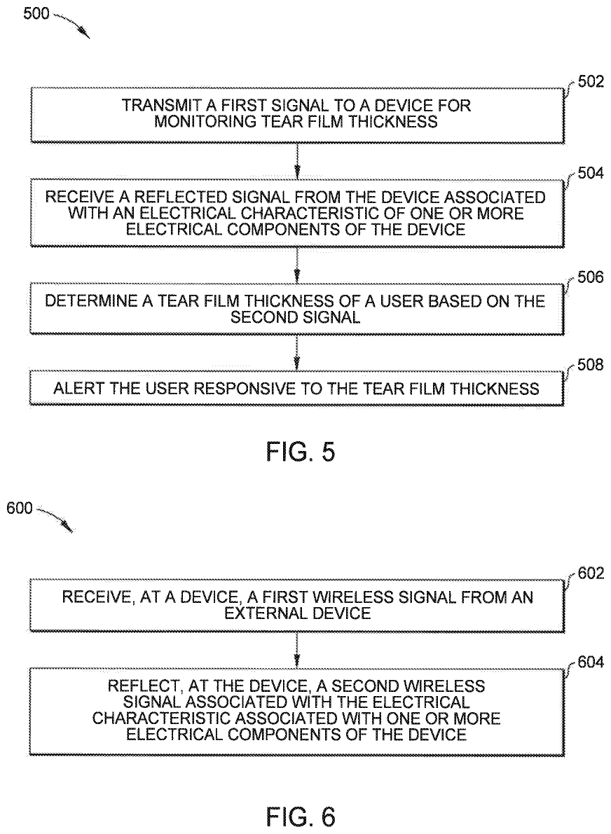

FIG. 4A illustrates an example system 420 for monitoring tear film thickness, according to certain embodiments. System 420 shows device 400 disposed on an eye 410. The device 400 may be any one of the devices 100, 200, and/or 300.

As shown in FIG. 4A, an external device 412 (described in further detail below with respect to FIG. 4B) is operable to transmit a first signal 414 towards the device 400 to induce power in passive components of the one or more electrical components of device 400 to activate the one or more electrical components. In some embodiments, the external device 412 may send the first signal 414 continuously, for example, in the increments of milliseconds, seconds, minutes, etc. For example, the first signal can be transmitted every 1, 5, 10, 20 milliseconds/seconds/minutes. The first signal 414 is a wireless signal, including, for example, radio frequency energy operable to activate passive components in the device 400.

When the first signal 414 reaches the one or more electrical components, a second signal 416 is transmitted back that is indicative of a measurement of the electrical characteristic of the one or more electrical components. As an example, in embodiments where the one or more electrical components include a temperature sensor, the temperature is measured responsive to energy received, from first signal 414, by passive components of the temperature sensor (e.g., passive RFID components) and a second signal 416 is transmitted back that is indicative of the temperature measurement. The external device 412 may then receive the second signal 416 from the electrical component and store the temperature measurement in memory. In some embodiments, the external device 412 may include, for example, a watch, phone, tablet, computer, or combinations of the same and like.

FIG. 4B illustrates an example external device 412, according to aspects of the disclosure. The external device 412 may include, for example, a computer, phone, tablet, and/or smartwatch, or other similar device, and may be operable to determine the tear film thickness of the eye based on at least one measurement of an electrical characteristic of one or more electrical components of a device (e.g., device 100, 200, 300) that can be disposed on an eye. As described above, the electrical characteristic may include the resonance frequency of an antenna, capacitance of a capacitive element, temperature of a temperature sensor, or the like. As illustrated in FIG. 4B, the external device 412 includes a central processing unit (CPU) 451, memory 452, storage 454, an input/output (I/O) interface 453, and transceiver 455 communicatively coupled via interconnect (bus) 456.

The CPU 451 may retrieve and store application data in the memory 452, as well as retrieve and execute instructions stored in the memory 452 and/or storage 454. The interconnect 456 transmits programming instructions and application data among the CPU 451, memory 452, storage 454, I/O interface 453, and transceiver 455. The CPU 451 can represent a single CPU, multiple CPUs, a single CPU having multiple processing cores, and the like. The memory 452 can represent random access memory. The storage 454 may be a disk drive. Although shown as a single unit, storage 454 may be a combination of fixed or removable storage devices. Memory 452 and/or storage 454 may include an operating system and/or one or more applications that, when executed by CPU 451, operate external device 412.

The storage 454 may include data and/or software usable to determine tear film thickness. In certain embodiments, the storage 454 can include, for example, a set of instructions for determining tear film thickness based on at least one measurement of an electrical characteristic of an electrical component. For example, the instructions may configure the CPU 451 to take one or more resonance frequency, capacitance, and/or temperature measurements and map them to a particular tear film thickness or change in tear film thickness, for example, using a defined mapping. In certain embodiments, the mapping is a table or a database of electrical characteristic measurements with the corresponding tear film thicknesses. In such embodiments, executing the instructions causes CPU 451 to take an electrical characteristic measurement or a change in the electrical characteristic from a device (e.g., device 100, 200, 300) as input and look up the corresponding tear film thickness. In certain embodiments, the mapping may be a function or an algorithm. In such an embodiment, executing the instructions causes CPU 451 to take an electrical characteristic measurement from a device (e.g., device 100, 200, 300) as input into the function, which will output a corresponding tear film thickness.

Similarly, various mappings between changes in an electrical characteristic and tear film thicknesses may be provided. In such embodiments, executing the instructions causes CPU 451 to take at least two electrical characteristic measurements (e.g., resonance frequencies at T0 and T1) from a device (e.g., device 100, 200, 300) as input, calculate a change in the electrical characteristic (e.g., resonance frequency shift from T0 to T1), and map it to a certain tear film thickness or change in tear film thickness.

In certain embodiments, the defined mappings described above may be based on empirical research involving a large user population. For example, it may be determined that for average users, certain resonance frequencies correspond to certain tear film thicknesses, or that certain capacitance values correspond to certain tear film thicknesses, or the link.

The I/O interface 453 includes hardware, software, or both, providing one or more interfaces for communication between the external device 412 and one or more I/O devices. External device 412 may be communicably connected to one or more of these I/O devices, which may be incorporated into, plugged into, paired with, or otherwise communicably connected to the external device 412. An input device may include any suitable device for converting user input into digital signals that may be processed by external device 412, such as, by way of example and not limitation, a touch screen and/or a keyboard. An output device may include any suitable device for converting output from the external device 412 such that the output may be accessible to the user (e.g., a display, a speaker, a haptic feedback device, etc.).

The transceiver 455 includes hardware, software, or both, that may transmit or receive communications to and/or from the external device 412. For example, the transceiver 455 may include a transceiver operable to send the first signal 414 and receive the second signal 416, as described above, to/from an electrical component. In certain embodiments, the transceiver 455 may include, for example, an NFC transceiver. In some embodiments, the transceiver 455 includes a networking interface operable to connect the external device 412 to a wireless and/or hardware network. Additionally and/or alternatively, the transceiver 455 may include a BLUETOOTH transceiver from communicating with an electrical component (e.g., the temperature sensor 310) or other device.

In some embodiments, external device 412 may store historical data relating to tear film thickness such as resonance frequency, capacitance, and/or temperature measurements collected over time. In some embodiments, the external device 412 may be operable to identify trends in the historical data and make recommendations to a user based on the identified trends. For example, if an identified trend shows a tear film thickness below an acceptable range between the hours of 9 AM and 5 PM, the external device 412 may prompt the user with advice to take breaks when using electronic devices for more than one hour. In another example, if an identified trend shows a tear film thickness below an acceptable range at a certain location, the external device 412 may prompt the user with the root cause of dry eyes of the user possibly being the location the user visits.

In certain embodiments, the historical data may be used to train machine learning models able to predict tear film thickness at a specific time of day. For example, the external device 412 may operate one or more algorithms using computational methods to learn user patterns and tear film thickness based on times of the day. In some embodiments, the tear film data used for computational analysis and/or learning may include data (e.g., time of day) that indicates a change in tear film thickness outside of an acceptable level. As more tear film data is received, the one or more algorithms may adaptively improve the performance. In certain embodiments, predictions related to user patterns and/or tear film thickness may be utilized to generate and provide notifications to a user to take appropriate action to prevent tear film thickness from falling outside of a desired range, thereby enabling proactive or preemptive treatment for dry eye or related conditions. For example, the external device may send a notification to a user to alert the user to apply eye drops prior to a time of day when the tear film thickness is predicted to fall outside of a desired range by the trained machine learning models.

In some embodiments, the historical data may be used to calibrate the device 300. For example, the external device 412 may utilize the historical data to build calibration curves of a sensor within the device monitoring tear film thickness. Subsequently, in some embodiments, the sensor may be calibrated with the external device 412 for each measurement along the calibration curve.

In some embodiments, the historical data may be uploaded to a doctor and/or clinician to assist in diagnosis of dry eye and/or diseases related to dry eye.

In some embodiments, the external device 412 may display the tear film thickness on a display of the external device 412. For example, the external device 412 may display the real-time tear film thickness or predicted tear film thickness for a projected period of time. In certain embodiments, the external device 412 may be further operable to alert a user responsive to the tear film thickness being below an acceptable range (e.g., below 3 μm) and/or outside a user-set range. In some embodiments, the alert may include a recommended course of action to prevent and/or alleviate dry eye (e.g., an alert to the user to apply eye drops). In some embodiments, the alert may include a prompt to remind the user to take a break from using electronic devices. In these and other embodiments, the alert may be proactive or preemptive. For example, it may be determined or known at what threshold a user begins to experience discomfort from dry eyes. This may be determined from studies or a large number of people more broadly, or from specific feedback from a specific user (e.g., the user may select an icon indicating they are experiencing discomfort due to dry eyes), or a combination of both. The external device 412 may alert the user to take a course of action (e.g., apply eye drops) at a first higher threshold before the tear film thickness reaches a second lower threshold.

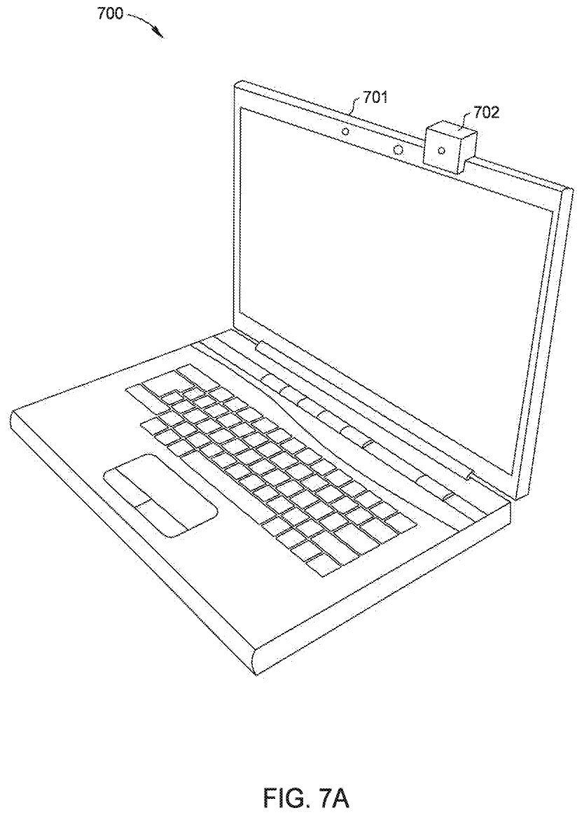

FIG. 5 illustrates an example method 500 for monitoring tear film thickness, according to certain embodiments. At step 502, an external device (e.g., external device 412) transmits a first signal to a device for monitoring tear film thickness (e.g., devices 100, 200, and/or 300). In certain embodiments, the transmission may occur over different communication protocols, such as, for example, high-frequency, ultrahigh-frequency, and/or near field communications protocols. In some embodiments, this includes an RFID signal.

At step 504, the external device receives a return signal from the device associated with an electrical characteristic of one or more electrical components of the device. In some embodiments, the external device may include an infrared thermometer such that temperature may be obtained with or without a device for monitoring tear film thickness in the eye, for example, by measuring the reflective energy and/or emitted energy by the eye.

At step 506, the external device determines a tear film thickness of a user based on the second signal. As described above, in certain embodiments, the external device determines the tear film thickness using mappings, including tables, databases, functions, algorithms, etc., to map the information provided by the second signal (e.g., a resonance frequency, capacitance, temperature, etc.) to a certain tear film thickness or change in tear film thickness. In certain embodiments, the determining may include predicting, using a machine learning model for example, a tear film thickness for a specific time and/or interval of time.

At step 508, the external device alerts the user responsive to the tear film thickness. For example, the user may be alerted if the tear film thickness is below an acceptable range or there is a significant change in the tear film thickness. In some embodiments, the alert may include alarms, notifications, haptic feedback, or combinations of the same and like. In certain embodiments, the acceptable range is user defined or based on a predicted acceptable tear film thickness range.

In some embodiments, steps of method 500 may be performed in parallel or in tandem. For example, the external device may transmit a first signal while determining tear film thickness based on a previous signal received. In certain embodiments, method 500 may be performed continuously and/or in real-time.

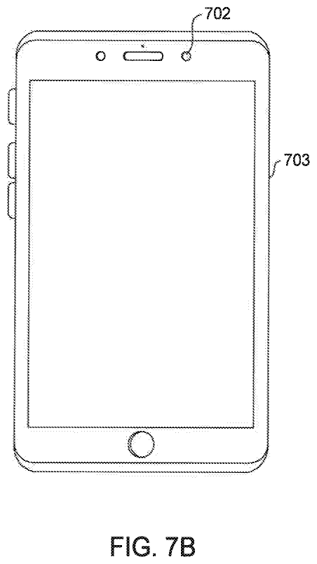

FIG. 6 describes a method of facilitating monitoring an electrical characteristic of an electrical component, the electrical characteristic associated with a tear film thickness of an eye. The method includes receiving a first wireless signal from an external device (step 602) and, responsive to the first wireless signal, transmitting a second wireless signal associated with the electrical characteristic to the external device (step 604). In certain embodiments, the first wireless signal may activate the electronic components by, for example, radio frequency energy.

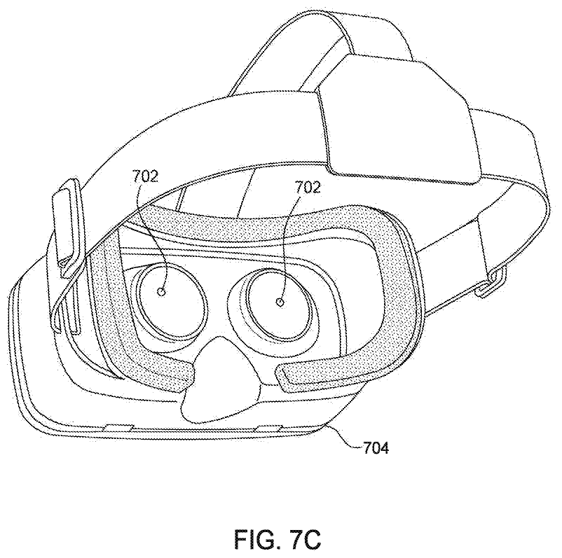

While the above embodiments are described relative to measuring tear film thickness utilizing a contact lens, other embodiments are readily envisioned. For example, FIG. 7A illustrates a tear film thickness monitoring system 700 having a computer 701 and an infrared (IR) temperature sensor 702. In some embodiments, the computer 701 may be representative of the external device 412 illustrated in FIG. 5. In embodiments illustrated in FIG. 7A, a user may have their tear film thickness monitored while using computer 701. For example, in some embodiments, while a user is using the computer 701, the IR temperature sensor 702 may monitor temperature of the eye continuously or periodically. In some embodiments, the IR temperature sensor 702 measures the reflective energy and/or emitted energy by the eye as the user looks at the screen of computer 701. As described above, evaporation of fluids in the eye may lead to temperature changes in the eye. In certain embodiments, the IR temperature sensor 702 may measure the temperature at the eye and detect the change in temperature over time. In certain embodiments, an application running on the computer 701 may map the change in temperature over time to, for example, a database value, to determine the thickness of the tear film. In some embodiments, the computer 701 may calculate the tear film thickness based on the change in temperature of the eye.

In certain embodiments, the IR temperature sensor 702 is communicatively coupled to the computer 701. In some embodiments, the IR temperature sensor 702 is coupled via a wired connection to the computer 701. In some embodiments, the IR temperature sensor 702 is coupled via a wireless connection (e.g., BLUETOOTH) to the computer 701. In some embodiments, the IR temperature sensor 702 may be embedded in, or integrated with, the computer 701. In some embodiments, the IR temperature sensor 702 may communicate with the computer 701 similarly to that as described above relative to FIGS. 1-6. In some embodiments, the computer is operable to periodically enable the IR temperature sensor 702. In such embodiments, when the IR temperature sensor 702 is enabled, the computer 701 may take temperature readings of the eye via the IR temperature sensor 702 and calculate tear film thickness based on a change in temperature measure by the IR temperature sensor 702.

While the tear film thickness monitoring system 700 in FIG. 7A is illustrated as using computer 701, similar systems are readily envisioned as illustrated in FIGS. 7B-7C. For example, FIG. 7B illustrates the IR temperature sensor 702 integrated into a mobile phone 703. In some embodiments, the mobile phone 703 may be representative of the external device 412 illustrated in FIG. 4A. In embodiments illustrated in FIG. 7B, a user may hold the mobile phone 703 up to the eye such that the IR temperature sensor 702 may measure temperature in the eye. In certain embodiments, the user may hold the mobile phone 703 up to the eye for a duration of time such that the IR temperature sensor 702 may take multiple readings to determine a temperature change in time. In some embodiments, the user may periodically measure temperature of the eye using the IR temperature sensor 702. In some embodiments, the mobile phone 703 may use previous temperature measurements to determine a change of temperature over time (e.g., from the previous measurement to the current measurement). In some embodiments, the mobile phone 703 may calculate, or look up, the tear film thickness based on the change in temperature of the eye. While FIG. 7B illustrates mobile phone 703, in certain embodiments, functionality of the mobile phone 703 may be replaced by a tablet computer, smart watch, other mobile computing device, or similar device. Additionally and/or alternatively, in some embodiments, the mobile phone 703 may utilize an external IR temperature sensor 702.

FIG. 7C illustrates an augmented reality and/or virtual reality (AR/VR) headset 704 having IR temperature sensors 702 disposed on lenses in the AR/VR headset 704. In some embodiments, the AR/VR headset 704 may be representative of the external device 412 illustrated in FIG. 4B. In embodiments illustrated in FIG. 7C, a user may have the change in temperature of the eye measured while using the AR/VR headset 704. In certain embodiments, the AR/VR headset 704 may display the current tear film thickness of the user in an augmented and/or virtual display provided by the AR/VR headset 704. In some embodiments, the AR/VR headset 704 may calculate, or look up, the tear film thickness of the user based on the change in temperature of the eye. In certain embodiments, the IR temperature sensor 702 may communicate with an external device, for example, a phone, tablet, computer, and/or smartwatch in a similar manner to that as described above, such that the external device may calculate, or look up, the tear film thickness of the user based on a change in temperature of the eye over time. In some embodiments, the AR/VR headset 704 is operable to periodically enable the IR temperature sensor 702. In such embodiments, when the IR temperature sensor 702 is enabled, the AR/VR headset 704 may take temperature readings from the IR temperature sensor 702 and calculate tear film thickness based on a change in temperature of the IR temperature sensor 702. In certain embodiments, the IR temperature sensor 702 may measure temperature in the eye continuously. In certain embodiments, the user can trigger a temperature reading periodically while using the AR/VR headset 704. While FIG. 7C illustrates AR/VR headset 704, in certain embodiments, the AR/VR headset 704 may be replaced by another wearable computing device, such as glasses, or another similar device.

The aforementioned embodiments may monitor tear level thickness on a more frequent basis than certain existing methods. Monitoring tear level thickness as described above may allow for real time alerts when a tear film thickness drops below a threshold thickness and/or volume, allowing a user to stop or prevent dry eye before irritation occurs. Historical data may be provided to a user such that tear film thickness over time may be monitored to identify a root cause associated with times of the day where dry eye develops, or potential times that would be optimal for the user to use topical treatments for the eye. While embodiments disclosed herein are described with passive electronic components, in certain embodiments, the electronic components may include a battery and/or other power source. In such embodiments, the electronic components may further include a transmitter operable to send and/or receive signals between external devices. Embodiments are therefore readily envisioned such that a first signal to activate passive electronic components may not be needed.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those of ordinary skill in the art may better understand the aspects of the disclosure. Those of ordinary skill in the art should appreciate that they may readily use the disclosure as a basis for designing or modifying other processes and structures for carrying out the same purposes and/or achieving the same advantages of the embodiments introduced herein. Those of ordinary skill in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments.

While the above detailed description has shown, described, and pointed out novel features as applied to various embodiments, it will be understood that various omissions, substitutions, and changes in the form and details of the embodiments illustrated can be made without departing from the spirit of the disclosure. As will be recognized, the various embodiments described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A device for facilitating monitoring of tear film thickness of an eye, the device comprising:
a lens; and
one or more electronic components coupled to the lens, the one or more electronic components operable to:
receive a first wireless signal from an external device; and
responsive to the first wireless signal, transmit a second wireless signal associated with an electrical characteristic of the one or more electrical components or indicative of a parameter of the eye, wherein the external device is operable to receive the second wireless signal and determine the tear film thickness based on the electrical characteristic or the parameter.

2. The device of claim 1, wherein the one or more electronic components are coupled to an outside surface of the lens.

3. The device of claim 1, wherein the one or more electronic components comprise an antenna.

4. The device of claim 3, wherein the antenna comprises a radio frequency identification (RFID) antenna.

5. The device of claim 3, wherein the electrical characteristic comprises a resonance frequency of the antenna.

6. The device of claim 3, wherein:
the one or more electronic components comprise a capacitive element; and
the electrical characteristic comprises capacitance.

7. The device of claim 3, wherein:
the one or more electronic components comprise a temperature sensor; and
the parameter comprises temperature at a surface of the eye.

8. The device of claim 1, wherein the first wireless signal is operable to activate the one or more electronic components.

9. A system for monitoring tear film thickness of an eye, the system comprising:
an external device operable to transmit a first wireless signal;
a lens; and
one or more electronic components coupled to the lens, the one or more electronic components operable to:
receive the first wireless signal from the external device; and
responsive to the first wireless signal, transmit a second wireless signal associated with an electrical characteristic of the one or more electrical components or indicative of a parameter of the eye to the external device, wherein the external device is operable to receive the second wireless signal and determine the tear film thickness based on the electrical characteristic or the parameter.

10. The system of claim 9, wherein the one or more electronic components comprise an antenna.

11. The system of claim 10, wherein the antenna comprises a radio frequency identification (RFID) antenna.

12. The system of claim 10, wherein the electrical characteristic comprises a resonance frequency of the antenna.

13. The system of claim 12, wherein the external device is further operable to:
receive the second wireless signal; and
determine the tear film thickness of the eye based on the second wireless signal.

14. The system of claim 10, wherein:
the one or more electronic components comprise a capacitive element; and
the electrical characteristic comprises capacitance.

15. The system of claim 14, wherein the external device is further operable to:
receive the second wireless signal; and
determine the tear film thickness of the eye based on the second wireless signal.

16. The system of claim 10, wherein:
the one or more electronic components comprise a temperature sensor; and
the parameter comprises temperature.

17. The system of claim 16, wherein the external device is further operable to:
receive the second wireless signal; and
determine the tear film thickness of the eye based on the second wireless signal.

18. A method of monitoring tear film thickness of an eye, the method comprising:
sending a first wireless signal to a device operable to facilitate monitoring the tear film thickness, the device comprising:
a lens; and
one or more electronic components coupled to the lens;
responsive to the first wireless signal, receiving a second wireless signal transmitted by the device, wherein the second wireless signal is associated with an electrical characteristic associated with the one or more electrical components or indicative of a parameter of the eye; and
determining the tear film thickness of the eye based on the second wireless signal.

19. The method of claim 18, wherein the electrical characteristic comprises at least one of a resonance frequency, capacitance, or temperature.

20. The method of claim 18, further comprising alerting a user responsive to the tear film thickness being below a defined range.
